(19) United States
(12) Patent Application Publication
(10) Pub. No.: US 2025/0198918 A1
(43) Pub. Date: Jun. 19, 2025

(54) SYSTEM AND METHOD FOR TERAHERTZ TOMOGRAPHY ANALYSIS OF GOLF BALLS

(71) Applicant: Acushnet Company, Fairhaven, MA (US)

(72) Inventor: Robert Blink, Newport, RI (US)

(73) Assignee: Acushnet Company, Fairhaven, MA (US)

(21) Appl. No.: 18/977,395

(22) Filed: Dec. 11, 2024

Related U.S. Application Data
(60) Provisional application No. 63/609,906, filed on Dec. 14, 2023.

(51) Int. Cl. G01N 21/3581; G01B 11/06; G01N 33/00

(52) U.S. Cl. CPC G01N 21/3581; G01B 11/0616; G01N 33/008

(57) ABSTRACT

A method, system, and techniques are disclosed herein that provides non-destructive analysis of a golf ball or golf equipment. More specifically, the present disclosure is related to using terahertz tomography to identify imperfections, missing layers, or other undesirable features of a golf ball construction, as well as identifying the composition of various golf ball components or layers. The present disclosure also provides for a relatively quick examination technique that can be implemented in-line with existing manufacturing lines due to its ability to rapidly scan the golf balls.

### INCORPORATION BY REFERENCE

The present application claims the benefit of U.S. Provisional Patent Application No. 63/609,906, filed on Dec. 14, 2023, the entire disclosure of which is hereby incorporated herein by reference.

### FIELD OF THE INVENTION

This disclosure generally relates to analysis of a golf ball, and more particularly relates to analysis of a golf ball via terahertz imaging and other techniques using terahertz tomography.

### BACKGROUND OF THE INVENTION

Analysis techniques for golf balls are well known. Some known techniques for analyzing golf balls include analysis via x-ray radiation. These systems typically use ionizing radiation and can alter the underlying structure of golf balls. Other known techniques that do not require ionizing radiation can be labor and/or time intensive.

It would be desirable to provide a golf ball analysis technique that addresses these issues.

### SUMMARY OF THE INVENTION

In some aspects, the present disclosure is directed to a method of detecting and/or analyzing at least one characteristic of a golf ball. The method can comprise acquiring imaging data related to at least one characteristic of a golf ball via a terahertz scanner assembly or other equipment configured to use terahertz imaging or tomography. In one specific aspect, the present disclosure is related to terahertz tomography analysis of golf ball layers, and more specifically can be directed to analysis of paint or coating layers of a golf ball, in one aspect. In another aspect, the present disclosure is related to using terahertz tomography or imaging for analysis of other components of a golf ball, such as a cover, a casing, or other layer of a golf ball. In one aspect, the present disclosure is related to terahertz time domain spectroscopy or tomography. In one aspect, the present disclosure provides improved imaging, process control, and quality assurance for golf ball manufacturing.

In one aspect, the method includes generating at least one parameter from the imaging data and comparing the at least one parameter against a threshold value. In another aspect, the method can include generating a notification if or when the at least one parameter exceeds the threshold value. The parameter(s) can include, for instance, measurements or values associated with a thickness, composition, chemical information (i.e., identity, ratio of chemical compounds, arrangement, etc.), concentricity, presence/absence of layers, etc. Various other parameters are disclosed herein.

The at least one characteristic of the golf ball can include, for example, at least one of: (i) a thickness of at least one paint or coating layer on the cover layer; (ii) a composition of at least one paint or coating layer on the cover layer; (iii) a presence of at least one paint or coating layer on the cover layer; (iv) a thickness of the cover layer; (v) a thickness of a casing layer surrounding the cover layer; (vi) a composition of the cover layer; or (vii) a composition of a casing layer surrounding the cover layer. One of ordinary skill in the art would understand that any one or more of the combinations of these characteristics can be detected or analyzed via the terahertz imaging analysis assembly and techniques disclosed herein.

In one aspect, the terahertz imaging analysis assembly and techniques disclosed herein can provide information or data regarding both: (i) a thickness of at least one paint or coating layer on the cover layer, and (ii) a composition of at least one paint or coating layer on the cover layer. In one aspect, the terahertz imaging analysis assembly and techniques disclosed herein can provide information or data regarding a presence of at least one paint or coating layer on the cover layer. In one aspect, the terahertz imaging analysis assembly and techniques disclosed herein can provide information or data regarding both: (i) a thickness of the cover layer, and (ii) a composition of the cover layer. In one aspect, the terahertz imaging analysis assembly and techniques disclosed herein can provide information or data regarding both: (i) a thickness of a casing layer surrounding the cover layer, and (ii) a composition of the casing layer surrounding the cover layer.

The at least one characteristic of the golf ball can include characteristics of a cover or casing of the golf ball, in another example. In another aspect, the characteristic can be associated with other layers of the golf ball, such as the core (or sub-layers of the core), any number of intermediate layers, and any number of cover layers. The at least one characteristic of the golf ball can include at least one of: (i) concentricity of at least two layers of the golf ball; (ii) thickness of at least one paint or coating layer; (iii) composition of at least one paint or coating layer; or (iv) presence of at least one paint layer.

In another aspect, the at least one characteristic of the golf ball includes all of the following: (i) concentricity of at least two layers of the golf ball; (ii) thickness of at least one paint or coating layer; (iii) composition of at least one paint or coating layer; and (iv) presence of at least one paint or coating layer. One of ordinary skill in the art would understand that the at least one characteristic can vary. Additionally, one of ordinary skill in the art would understand that the techniques disclosed herein can be used on any feature of a golf ball, including but not limited to coating layers, paint layers, cover layers, casing layers, or other aspects of a golf ball.

The terahertz scanner assembly can include multiple terahertz scanners that are each arranged at a different orientation relative to the golf ball or golf balls, in one aspect. For example, the terahertz scanner assembly can comprise at least one first terahertz scanner arranged at a first angle relative to the golf ball and at least one second terahertz scanner arranged at a second angle relative to the golf ball. The first angle can be different than the second angle.

The method can further comprise spinning or otherwise rotating or manipulating the golf ball during analysis or emission of the terahertz radiation. One of ordinary skill in the art would understand that a spindle or other support assembly could be used to hold and rotate the golf ball. In one aspect, the terahertz scanner can move relative to a golf ball.

In one aspect, the method of analyzing the golf ball is non-destructive. In one aspect, the method of analyzing the golf ball employs non-ionizing radiation. Accordingly, the golf ball remains completely intact and unchanged during the analysis.

In one aspect, acquiring the imaging data takes less than 100 nanoseconds. In another aspect, acquiring the imaging data takes less than 10 nanoseconds.

An imaging area of the terahertz scanner assembly can be configured to be focused only on a periphery of the golf ball. The periphery of the golf ball can only include a portion of at least one paint or coating layer, in one aspect. In another aspect, the periphery of the golf ball can only include a portion of at least one paint or coating layer, and a portion of a cover. In another aspect, the periphery of the golf ball can only include a portion of at least one paint or coating layer, a portion of a cover, and a portion of a casing.

The terahertz scanner assembly can have a resolution of at least 0.1 micron-25.0 microns. In one aspect, the resolution can be less than 0.1 micron or greater than 25.0 microns. The terahertz scanner assembly can be configured to emit terahertz radiation at a frequency of 0.1 terahertz-10.0 terahertz. In one aspect, the frequency can be less than 0.1 terahertz or greater than 10.0 terahertz.

In another aspect, a method of detecting and/or analyzing at least one characteristic of a golf ball via non-ionizing electromagnetic radiation is provided. The method can include directing an imaging area of a terahertz scanner assembly at a periphery of the golf ball. The periphery of the golf ball can only include a portion of at least one paint or coating layer, and a portion of a cover, in one aspect. The method can further include acquiring imaging data related to at least one characteristic of a golf ball via the terahertz scanner assembly. In one aspect, the at least one characteristic of the golf ball includes at least one of: (i) a thickness of at least one paint or coating layer on the cover layer; (ii) a composition of at least one paint or coating layer on the cover layer; (iii) a presence of at least one paint or coating layer on the cover layer; (iv) a thickness of the cover layer; (v) a thickness of a casing layer surrounding the cover layer; (vi) a composition of the cover layer; or (vii) a composition of a casing layer surrounding the cover layer. Various other aspects of the present disclosure are provided in more detail herein.

### BRIEF DESCRIPTION OF THE DRAWINGS

Further features and advantages of the present disclosure can be ascertained from the following detailed description that is provided in connection with the drawings described below:

FIG. 1 is schematic illustration of a system including at least one terahertz scanner assembly for analyzing golf balls.

FIG. 2 is a magnified view of an imaging area for the terahertz scanner assembly focused on a portion of a golf ball.

FIG. 3 is a view of a support assembly for a golf ball relative to the terahertz scanner assembly.

### DETAILED DESCRIPTION OF THE INVENTION

According to disclosed embodiments, an improved method, system, and configuration are provided herein that generates and provides data or information regarding a golf ball. More specifically, the present disclosure is directed to an analysis and imaging technique that uses terahertz radiation to obtain information regarding a golf ball construction. In one aspect, the imaging techniques provided herein are configured to provide information regarding certain qualities of layers of a golf ball, such as paint layers or coating layers. For example, the quality of an applied paint or coating layer can be analyzed using the techniques disclosed herein. In one aspect, the presence or absence of a layer on the golf ball, such as a paint or coating layer, can be determined using the techniques disclosed herein. In other aspect, the present disclosure provides for analysis of concentricity of a golf ball construction. In another aspect, the present disclosure is directed to providing information, images, or data regarding any layer's thickness, composition, presence or absence, quality, and/or consistency. The present disclosure can be configured to identify layers that are thicker or thinner than a target thickness, contain imperfections, and/or identify any missing coatings or layers. In one aspect, the analysis disclosed herein can identify and analyze any contaminants that may be present within certain layers of the golf ball. The present disclosure can also be configured to provide information regarding the chemical composition of any layers, such as paint or coating layers, or a cover, casing, or core, of the golf ball. Regarding the chemical composition analysis, the present disclosure can provide the ability to analyze the concentration, presence, and/or other characteristics of various materials used in golf ball layers, paint layers, coating layers, or other aspects of the golf ball. In one aspect, the chemical composition analysis disclosed herein can provide the ability to verify that a golf ball color meets a predetermined threshold, includes certain levels of concentrations of colorants, or other measurements regarding colorants and/or color characteristics. The techniques disclosed herein can be used to measure or detect compounds that affect color or contribute to color properties.

In one aspect, the analysis or measurements of the golf ball can occur after casing molding, cover casting/molding, or painting to determine the thickness or other characteristics of the layers and sub-layers. In one aspect, the techniques disclosed herein can be carried out on any one or more components of the golf ball, such as the core, cased core, etc. One of ordinary skill in the art would understand that the analysis of the golf ball can occur at any stage of manufacturing the golf ball. The imaging equipment (also referred to herein as imagers, detectors, scanners, terahertz scanner assembly, etc.) can be disposed along the manufacturing line to scan each golf ball as the golf ball travels through a specific region of the manufacturing line. The present disclosure can be configured to sort golf balls along the manufacturing line and divert golf balls and/or provide an alert or indication of golf balls that fail a predetermined testing rubric using data or information generated by the terahertz scanner assembly.

In some embodiments, the present disclosure is directed to a method of detecting and/or analyzing at least one characteristic of a golf ball. The method can comprise acquiring imaging data or information related to at least one characteristic of a golf ball via a terahertz scanner assembly. The term characteristic can be used herein to refer to various parameters, features, traits, qualities, or other properties of a golf ball.

In one example, the at least one characteristic of the golf ball can include at least one of: (i) a thickness of at least one paint or coating layer; (ii) a composition of at least one paint or coating layer; (iii) a presence of at least one paint or coating layer; (iv) a thickness of a cover layer; (v) a thickness of a casing layer; (vi) a composition of a cover layer; or (vii) a composition of a casing layer. In one aspect, the thickness of the paint or coating layer can be analyzed to determine if any portion of the layer is inconsistent. Various metrics can be utilized to determine the consistency of paint or coating layers, as one of ordinary skill in the art would appreciate based on the present disclosure.

The at least one characteristic of the golf ball can include at least one characteristic of a cover or casing of the golf ball. In another aspect, the characteristic can be associated with other layers of the golf ball, such as core (or sub-layers of the core), any number of intermediate layers, and any number of cover layers. In one example, the at least one characteristic of the golf ball can include at least one of: (i) a thickness of at least one paint or coating layer on the cover layer; (ii) a composition of at least one paint or coating layer on the cover layer; (iii) a presence of at least one paint or coating layer on the cover layer; (iv) a thickness of the cover layer; (v) a thickness of a casing layer surrounding the cover layer; (vi) a composition of the cover layer; or (vii) a composition of a casing layer surrounding the cover layer. In another aspect, the at least one characteristic of the golf ball includes all of the following: (i) concentricity of at least two layers of the golf ball; (ii) thickness of at least one paint or coating layer; (iii) composition of at least one paint or coating layer; and (iv) presence of at least one paint or coating layer. One of ordinary skill in the art would understand that the at least one characteristic can vary.

The terahertz scanner assembly can include multiple terahertz scanners that are each arranged at a different orientation relative to the golf ball, in one aspect. For example, the terahertz scanner assembly can comprise at least one first terahertz scanner arranged at a first angle relative to the golf ball and at least one second terahertz scanner arranged at a second angle relative to the golf ball. The first angle can be different than the second angle. In one aspect, the first and second angles are anywhere between 1 degree apart −359 degrees apart. In one aspect, the first and second angles are 30 degrees apart, 45 degrees apart, 60 degrees apart, 75 degrees apart, 90 degrees apart, 105 degrees apart, 120 degrees apart, 135 degrees apart, 150 degrees apart, 165 degrees apart, or 180 degrees apart. Multiple scanner assemblies can be arranged at various spacings around a subject golf ball. In one aspect, a composite image can be generated based on a plurality of images from one or more terahertz scanner assemblies.

In one aspect, the golf ball can undergo analysis while being transported along a manufacturing line. In another aspect, the golf ball can undergo analysis while being transported along the manufacturing line and simultaneously rotated.

In one aspect, the method can further comprise spinning the golf ball. One of ordinary skill in the art would understand that a spindle or other support assembly could be used to hold, transport, and/or rotate the golf ball.

In one aspect, the method of analyzing the golf ball is non-destructive. In one aspect, the method of analyzing the golf ball employs non-ionizing radiation. The inspection or analysis techniques disclosed herein do not require any potentially damaging or structurally altering impacts on the golf ball. In one aspect, the present disclosure explicitly does not require exposure to x-ray radiation, dissecting, cutting, or otherwise damaging the golf balls.

In one aspect, acquiring the imaging data takes less than 100 nanoseconds. In another aspect, acquiring the imaging data takes less than 10 nanoseconds. In another aspect, acquiring the imaging data takes less than 1.0 nanoseconds. In another aspect, acquiring the imaging data takes less than 0.1 nanoseconds. In yet another aspect, acquiring the imaging data takes less than 0.01 nanoseconds.

In one aspect, the analysis technique may acquire imaging data over a longer period of time. In another aspect, imaging data can be obtained over 100 microseconds or more. In yet another aspect, data obtained over 1 second, or longer, can be obtained. In one aspect imaging data can be acquired over a period of minutes.

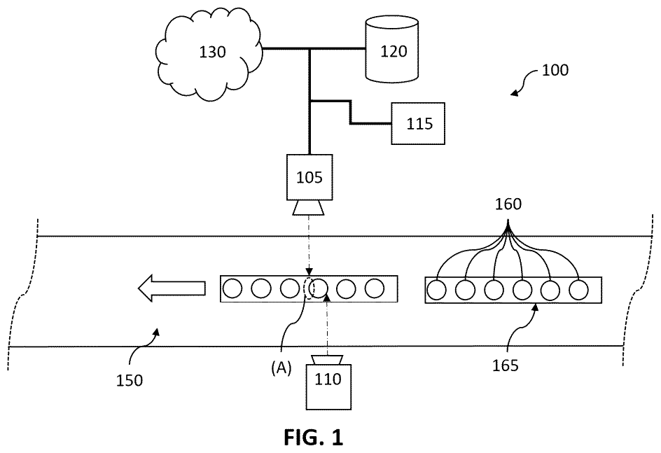

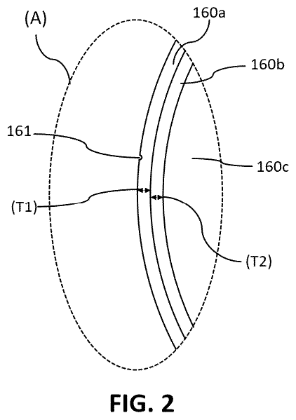

An imaging area, which is annotated as imaging area (A) in FIGS. 1 and 2, of the terahertz scanner assembly can be configured to be focused only on a periphery of the golf ball. The method can include directing or aiming the terahertz scanner assembly towards the golf ball such that an imaging area is focused on a periphery of the golf ball.

The periphery of the golf ball can only include a portion of at least one paint or coating layer, in one aspect. In another aspect, the periphery of the golf ball can only include a portion of at least one paint or coating layer, and a portion of a cover. In another aspect, the periphery of the golf ball can only include a portion of at least two paint or coating layers, and a portion of a cover. In another aspect, the periphery of the golf ball can only include a portion of at least two paint or coating layers, a portion of a cover, and a portion of a casing.

The terahertz scanner assembly can have a resolution of at least 0.1 micron-25.0 microns. In one aspect, the resolution can be less than 0.1 micron or greater than 25.0 microns. In one aspect, the resolution can be 0.01 micron-1.0 micron.

The at least one terahertz scanner assembly can be configured to emit electromagnetic radiation at a frequency of 0.1 terahertz-10.0 terahertz. In one aspect, the frequency can be less than 0.1 terahertz or greater than 10.0 terahertz.

One of ordinary skill in the art would understand that multiple imaging assemblies could provide data or information regarding parameters or characteristics of the golf ball, such as concentricity, consistency, etc., from multiple angles.

The imaging equipment disclosed herein can operate on a pico-second or nano-second scale, and therefore can be configured to quickly capture or generate multiple images using a single scanner assembly by spinning the golf ball during measurement or analysis.

In one aspect, the method includes generating at least one parameter from the imaging data and comparing the at least one parameter against a threshold value. In another aspect, the method can include generating a notification if or when the at least one parameter exceeds the threshold value. The parameter can include a numeral or calculation that is generated based on the imaging data, in one example. The parameter can be automatically generated or detected based on known imaging analysis tools.

A system 100 is shown in FIG. 1 that includes at least one terahertz scanner assembly 105, 110. As shown in FIG. 1, a first terahertz scanner assembly 105 is oriented or positioned in a first configuration and a second terahertz scanner assembly 110 is oriented or positioned in a second configuration that is different than the first configuration. The various terahertz scanner assemblies can be positioned on different sides of a manufacturing line 150 that is configured to convey or otherwise move golf balls 160 (the conveying direction being indicated in FIG. 1 by an enlarged arrow). In another example, more than one terahertz scanner assembly can be positioned on the same side of the manufacturing line 150. In another example, one of the terahertz scanner assemblies can be positioned above the manufacturing line 150, or in any another position.

As shown in FIG. 1, the system 100 can include a computer or any other electronics equipment, such as a central processing unit (CPU) or processor 115, a memory unit 120, and a server or cloud computing module 130. One of ordinary skill in the art would understand that various other electronic components or devices can be used within the system 100. In one aspect, data or information from the terahertz scanner assembly 105, 110 is configured to be transmitted and received relative to the electronic components, such as the CPU or processor 115, memory unit 120, and server or cloud computing module 130. A user interface, which can include a display, can be implemented with the CPU or processor 115 and can be configured to allow personnel to manually or automatically interact with data or information generated via the at least one terahertz scanner assembly 105, 110. In one aspect, analysis parameters can be implemented via the CPU or processor 115 such that each golf ball 160 that is analyzed via the terahertz scanner assembly 105, 110 can be checked against various quality control standards. Golf balls 160 that fail any quality control or other testing protocols can be diverted from the manufacturing line 150 or can otherwise be flagged for remedial or corrective action.

In one aspect, the CPU or processor can be configured to generate at least one parameter from the imaging data and compare the at least one parameter against at least one threshold value. In one aspect, the CPU or processor can be configured to generate a notification when the at least one parameter exceeds the at least one threshold value. The CPU or processor can include a controller or control unit configured to execute various commands and/or analysis associated with the data or information generated via the system. The memory unit can be configured to store commands, execution code, algorithms, etc., associated with carrying out the various steps and processes disclosed herein.

As shown in FIG. 1, a holder assembly 165 can be provided that is configured to hold one or more golf balls 160. The holder assembly 165 can be formed from a material that is specifically designed so as to not interfere or otherwise obfuscate the analysis performed via the terahertz scanner assembly 105, 110.

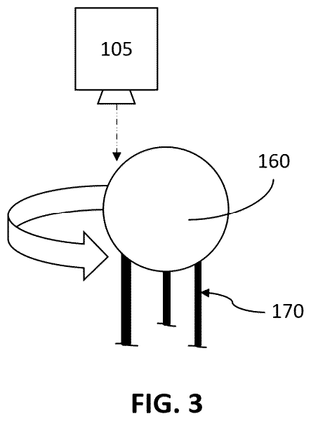

As shown in FIG. 3, in another aspect, a spindle assembly 170 can be provided that is configured to rotate the golf ball 160. In one aspect, the golf ball 160 can be conveyed along the manufacturing line 150 until it is positioned in a specific target area near the terahertz scanner assembly 105. Once the golf ball 160 is in the target area, any conveying motion of the golf ball 160 can be temporarily paused for a relatively short time (i.e., less than 1.0 nanosecond or less than 0.1 nanosecond) while the terahertz scanner assembly 105 emits radiation and performs its scanning or imaging process.

In one aspect, image analysis can be performed based on analysis of the pixels as captured or obtained via the scanner assembly. For example, various image analysis software or algorithms can be configured to process at least one image or a plurality of images obtained via the scanner assembly.

As shown in FIG. 1, the terahertz scanner assembly 105 is focused on or targeting a specific region of the golf ball, which is referred to herein as the imaging area (A) and is shown in FIG. 1. FIG. 2 shows a magnified view of the imaging area (A). In one example, as shown in FIG. 2, the imaging area (A) can be focused on a first paint or coating layer 160a, a second paint or coating layer 160b and a cover 160c of the golf ball 160. Other configurations, such as a single paint or coating layer, or more than two paint or coating layers, etc., can be analyzed using the techniques in the present disclosure. In other aspects, characteristics of the cover 160c, or any underlying layers, such as intermediate layers, casing layers, core layers, etc., can be analyzed.

The imaging area (A) can be modified to be larger, smaller, or in a different region relative to the subject golf ball, as one of ordinary skill in the art would appreciate based on this disclosure. In another aspect, multiple imaging areas can be provided on a single golf ball via multiple terahertz scanner assemblies.

As shown in FIG. 2, an imperfection 161 is defined in the first paint or coating layer 160a, which can be configured to be detected or identified via the terahertz scanner assembly 105. The imperfection 161 can be an inconsistency in the paint or coating layer or other type of flaw. The imperfection 161 is provided for illustrative purposes only, and one of ordinary skill in the art would understand that various imperfections of any quantity, size, shape, dimension, chemical composition, etc., can be detected via the system 100. One of ordinary skill in the art would understand that the imperfection could be manifested on other layers of the golf ball, such as on the casing layer or other intermediate layer as well as the core. Additionally, imperfections can be manifested at an interface between adjacent layers. The present disclosure provides the ability to identify and analyze said imperfections.

In one aspect, a process, algorithm, or other methodology can be used such that a threshold area or size is set for imperfection analysis. For example, an imperfection threshold value can be set to be a predetermined value, such as n-number of pixels or a predetermined area in square microns or square millimeters. If any imperfections are detected within the golf ball layers of a size that exceeds the imperfection threshold value, then the system can generate an alert or other information or otherwise trigger some remedial action. If imperfections are detected within the golf ball layers that do not exceed the imperfection threshold value, then the system can be configured to process the golf balls according to ordinary handling or processing steps.

As shown in FIG. 2, a first thickness (T1) is annotated for the first paint or coating layer 160a and a second thickness (T2) is annotated for the second paint or coating layer 160b. The system 100 disclosed herein can be configured to measure, analyze, scan, or otherwise provide data or information regarding the first thickness (T1) and/or second thickness (T2), in one aspect. These thicknesses (T1, T2) can be compared to threshold thicknesses to identify paint or coating layers that do not meet a prerequisite criteria. In one aspect, the system 100 disclosed herein is configured to generate or provide information regarding any inconsistencies in the first paint or coating layer 160a and/or second paint or coating layer 160b.

In one aspect, various sensors and other components can be provided to ensure that the golf ball is positioned in the correct position relative to the terahertz scanner assembly. For example, a conveying system can include conveying means for transporting the golf balls along an assembly line. Sensors or other components can be provided to slow or stop the conveying means as the golf ball approaches the terahertz scanner assembly. Various components, such as timers, sensors, or controllers, can be implemented such that the golf ball remains stationary just long enough to undergo analysis via the terahertz scanner assembly. In some aspects, the ball can be rotated during the conveyer's stationary phase. If the golf ball does not pass any quality testing or assurance protocols, the golf ball can be removed from the remainder of golf balls along the assembly line, or otherwise be handled differently.

In one aspect, the present disclosure is only configured to provide analysis of outermost regions of the golf balls. In another aspect, the present disclosure can be configured to provide analysis of inner layers of the golf balls.

In one aspect, the present disclosure provides a non-destructive analysis methodology and system for determining the density of particular regions or areas of a golf ball. Accordingly, the present disclosure can provide the ability to detect non-uniform application of layers, such as paint or coating layers, on the golf ball surface.

In one aspect, the present disclosure provides an “in-line” (i.e., in situ with the existing manufacturing line) solution for analyzing at least one characteristic of a golf ball layer, such as a paint or coating layer. The golf balls can be analyzed without requiring removal of the golf balls from the manufacturing line, or relatively time-consuming analysis or scanning techniques.

In one aspect, analysis of the golf ball characteristics can be based on a single image or scan from the terahertz scanner assembly. In other aspects, analysis of the golf ball characteristics can be based on at least 50 images or scans from the terahertz scanner assembly. In other aspects, analysis of the golf ball characteristics can be based on at least 360 images or scans from the terahertz scanner assembly. One of ordinary skill in the art would understand that the quantity of scans or images required for analysis can vary. Various images can be combined with each other to provide a composite or additive profile of the golf ball.

In one aspect, a plurality of images can be generated via the system disclosed herein such that a three-dimensional model can be generated for any given golf ball. The three-dimensional model can provide data or information regarding the overall quality of the golf ball layers.

In one aspect, data or information generated via the terahertz scanner assembly can be compared against a threshold value. For example, if the thickness of any paint or coating layer deviates more than 0.1% from a threshold value, or more than 0.01% from a threshold value, or more than 0.001% from a threshold value, then a quality control module can flag the golf ball or otherwise provide an alert or notification for subsequent remedial or corrective action. The threshold value can correspond to thickness, in one aspect. In another aspect, the threshold value can correspond to concentricity. In another aspect, the threshold value can correspond to a compositional parameter.

One of ordinary skill in the art would understand that similar threshold limits can be set for the imperfection threshold value disclosed herein.

One of ordinary skill in the art would understand that other threshold or quality control modules can be configured to identify golf balls or otherwise provide alerts or notifications for golf balls that are identified as being outside the prerequisite specifications. One of ordinary skill in the art would understand that automation modules can be implemented that can be configured to provide alerts or notifications, and/or take remedial or corrective action without user interaction. A sorting module or apparatus can be configured to automatically remove golf balls that fail any tests.

In one aspect, a quality control or assurance system and/or methodology is disclosed herein for golf balls. The quality control or assurance system and/or methodology utilizes terahertz scanning technology to analyze golf balls. In one aspect, the quality control or assurance system and/or methodology can be configured to ascertain whether golf balls have any imperfections or elements that would render the golf balls outside the limits for a commercially acceptable golf ball.

In one aspect, a machine learning module can be implemented with any one or more of the systems or methods disclosed herein. For example, a machine learning module can be implemented such that images, data, or other information obtained via the system or methods disclosed herein can be stored and analyzed to further train a machine learning module to detect imperfections, variants, out of specification parameters, or other details regarding golf balls being analyzed via the method or system.

In one aspect, the present disclosure can obtain information or data regarding golf ball layers or paint or coating layers on a golf ball surface without requiring the use of fillers or other additives to the material or compositions ordinarily used to make a golf ball.

In another aspect, fillers, such as targeted fillers improving contrast for terahertz image analysis, can be added to any one or more components of the golf ball.

In one aspect, a method or system can be provided that includes both a terahertz scanning system as well as a secondary analysis assembly. In one aspect, the terahertz scanning system can be configured to analyze or image relatively outer surfaces or layers of the golf ball. In one aspect, the secondary analysis assembly can be configured to analyze or image relative inner surfaces or layers of the golf ball. The terahertz scanning system and the secondary analysis assembly can be configured to carry out or perform relative analysis procedures or steps simultaneously with each other such that data or information regarding the outer and inner layers or portions of the golf ball can be obtained at the same time, thereby providing manufacturing efficiencies. In one aspect, the secondary analysis assembly can use an electromagnetic energy source, such as x-rays. In another aspect, the secondary analysis assembly can use terahertz electromagnetic radiation. One of ordinary skill in the art would understand that the type of energy or technique used by the secondary analysis assembly can vary.

In one aspect, the present disclosure is directed to durability analysis of a golf ball via terahertz scanner assembly. Golf balls can be analyzed via a terahertz scanner assembly as disclosed herein in order to identify the specific location and/or cause of failure. The analysis techniques disclosed herein can be used to identify fractures or other failure modes associated with durability issues. A terahertz scan performed on a golf ball could also indicate the presence of deformities in sub-layers that are not otherwise visible and could lead to durability problems in use.

In one aspect, any one of the methods or systems disclosed herein can be configured to analyze other types of equipment or components, such as other sports equipment. In another aspect, any one of the methods or systems disclosed herein can be configured to analyze golf clubs, such as a golf club shaft or golf club head. In one aspect, the methods or systems disclosed herein can provide the ability to analyze the composition of various aspects of the golf club. In another aspect, the methods or systems disclosed herein can provide the ability to analyze grooves and other features typically defined on the face of a golf club head. In one aspect, the techniques disclosed herein can be used to measure coatings or other finishing layers applied to a golf club component. One of ordinary skill in the art would understand that any of the characteristics or other properties or parameters that are disclosed herein with respect to golf balls can similarly be analyzed for golf clubs.

One of ordinary skill in the art would understand that various terahertz scanning systems that are commercially available could be implemented according to the present disclosure. Exemplary terahertz scanners can include terahertz scanners that are commercially available via Boston Electronics Corporation, TeraSense Group, INO, Advantest Corporation, among other companies.

Any one or more of the systems or methods disclosed herein can employ commercially available image analysis systems or processes, such as Halcon from MVTec, MIL by Matrox, Cognex VisionPro, MATLAB, or other known software, algorithms, or solutions.

While it is apparent that the illustrative embodiments of the invention disclosed herein fulfill the objectives stated above, it is appreciated that numerous modifications and other embodiments may be devised by those skilled in the art. Therefore, it will be understood that the appended claims are intended to cover all such modifications and embodiments, which would come within the spirit and scope of the present invention.

What is claimed is:

1. A method of analyzing at least one characteristic of a golf ball comprising at least a core and a cover layer, the method comprising:
acquiring imaging data related to at least one characteristic of a golf ball via at least one terahertz scanner assembly; and
generating at least one parameter from the imaging data and comparing the at least one parameter against a threshold value,
wherein the at least one characteristic of the golf ball includes at least one of:
(i) a thickness of at least one paint or coating layer on the cover layer;
(ii) a composition of at least one paint or coating layer on the cover layer;
(iii) a presence of at least one paint or coating layer on the cover layer;
(iv) a thickness of the cover layer;
(v) a thickness of a casing layer surrounding the cover layer;
(vi) a composition of the cover layer; or
(vii) a composition of a casing layer surrounding the cover layer.

2. The method according to claim 1, wherein the at least one terahertz scanner assembly comprises a plurality of terahertz scanners that are each arranged at a different orientation relative to the golf ball.

3. The method according to claim 1, wherein the at least one terahertz scanner assembly comprises at least one first terahertz scanner arranged at a first angle relative to the golf ball and at least one second terahertz scanner arranged at a second angle relative to the golf ball, wherein the first angle is different than the second angle.

4. The method according to claim 1, the method further comprising spinning the golf ball.

5. The method according to claim 1, wherein the method is non-destructive relative to the golf ball, and the method employs non-ionizing radiation.

6. The method according to claim 1, further comprising generating a notification when the at least one parameter exceeds the threshold value.

7. The method according to claim 1, wherein acquiring the imaging data requires less than 10 nanoseconds.

8. The method according to claim 1, wherein an imaging area of the at least one terahertz scanner assembly is configured to be focused only on a periphery of the golf ball.

9. The method according to claim 8, wherein the periphery of the golf ball only includes a portion of at least one paint or coating layer.

10. The method according to claim 8, wherein the periphery of the golf ball only includes a portion of at least one paint or coating layer, and a portion of the cover layer.

11. The method according to claim 8, wherein the periphery of the golf ball only includes a portion of at least one paint or coating layer, a portion of the cover layer, and a portion of a casing layer surrounding the cover layer.

12. The method according to claim 1, wherein the at least one terahertz scanner assembly is configured to detect imperfections in an outermost paint or coating layer of the golf ball.

13. The method according to claim 1, wherein the at least one characteristic of the golf ball includes both the thickness of the at least one paint or coating layer on the cover layer, and the composition of the at least one paint or coating layer on the cover layer.

14. The method according to claim 1, wherein the at least one characteristic of the golf ball includes both the thickness of the cover layer, and the composition of the cover layer.

15. The method according to claim 1, wherein the at least one characteristic of the golf ball includes both the thickness of the casing layer, and the composition of the casing layer.

16. A method of analyzing at least one characteristic of a golf ball via non-ionizing electromagnetic radiation, the golf ball comprising at least a core and a cover layer, the method comprising:
directing an imaging area of at least one terahertz scanner assembly at a periphery of the golf ball, wherein the periphery of the golf ball only includes a portion of at least one paint or coating layer surrounding the cover layer, and a portion of the cover layer;
acquiring imaging data related to at least one characteristic of a golf ball via the at least one terahertz scanner assembly, wherein the at least one characteristic of the golf ball includes at least one of:
(i) a thickness of at least one paint or coating layer on the cover layer;
(ii) a composition of at least one paint or coating layer on the cover layer;
(iii) a presence of at least one paint or coating layer on the cover layer;
(iv) a thickness of the cover layer;
(v) a thickness of a casing layer surrounding the cover layer;
(vi) a composition of the cover layer; or
(vii) a composition of a casing layer surrounding the cover layer;
generating at least one parameter from the imaging data and comparing the at least one parameter against at least one threshold value; and
generating a notification when the at least one parameter exceeds the at least one threshold value.

17. The method according to claim 16, wherein the at least one characteristic of the golf ball includes both the thickness of the at least one paint or coating layer on the cover layer, and the composition of the at least one paint or coating layer on the cover layer.

18. The method according to claim 16, wherein the at least one characteristic of the golf ball includes both the thickness of the cover layer, and the composition of the cover layer.

19. The method according to claim 16, wherein the at least one characteristic of the golf ball includes both the thickness of the casing layer and the composition of the casing layer.

20. The method according to claim 16, wherein the at least one characteristic of the golf ball includes only the presence of the at least one paint or coating layer on the cover layer.
